(19) United States
(12) Patent Application Publication
(10) Pub. No.: US 2023/0358741 A1
(43) Pub. Date: Nov. 9, 2023

(54) METHOD FOR PREPARING DUAL-CHANNEL VISUAL MULTICOLOR FLUORESCENT PROBE AND DETECTION METHOD

(71) Applicant: HENAN POLYTECHNIC UNIVERSITY, Jiaozuo (CN)

(72) Inventors: Jun XU, Jiaozuo (CN); Lei JIA, Jiaozuo (CN); Rui LI, Jiaozuo (CN); Yongxin LI, Jiaozuo (CN)

(73) Assignee: HENAN POLYTECHNIC UNIVERSITY, Jiaozuo (CN)

(21) Appl. No.: 18/217,635

(22) Filed: Jul. 3, 2023

Related U.S. Application Data
(63) Continuation of application No. PCT/CN2023/082699, filed on Mar. 21, 2023.

(30) Foreign Application Priority Data
Mar. 28, 2022 (CN) 202210313892.5

(51) Int. Cl. G01N 33/569; G01N 21/64; G01N 33/533; G01N 33/94; C09K 11/65; C09K 11/02

(52) U.S. Cl. CPC G01N 33/56911; G01N 21/6428; G01N 33/533; G01N 33/9446; C09K 11/65; C09K 11/02; G01N 2333/32; B82Y 20/00

(57) ABSTRACT

Provided is a dual-channel fluorescence sensor based on in-situ synthesis of carbon dots on halloysite nanotubes (HNT) and loaded with a lanthanide metal-organic framework, which can implement rapid and simultaneous visual detection of DPA and TC. By using methods for preparing and using a dual-channel visual multicolor fluorescent probe above, the sensor has high stability and sensitivity, and is conducive to quick, accurate and intuitive detection of a biomarker.

### CROSS REFERENCE TO THE RELATED APPLICATIONS

This application is a continuation application of International Application No. PCT/CN2023/082699, filed on Mar. 21, 2023, which is based upon and claims priority to Chinese Patent Application No. 202210313892.5, filed on Mar. 28, 2022, the entire contents of which are incorporated herein by reference.

### TECHNICAL FIELD

The present invention relates to the technical field of nano-composite material preparation, in particular to a method for preparing a dual-channel visual multicolor fluorescent probe and a detection method.

### BACKGROUND

In recent years, various analytical methods have been developed to detect tetracycline (TC) and Bacillus anthracis (DPA).

Overuse and abuse of TC are caused due to its low cost, absorbability and broad-spectrum bacteriostasis, and result in a vast amount of residues in food and agricultural and animal husbandry production, which poses risks to human health once such foods are eaten. Traditional TC detection methods include high performance liquid chromatography, capillary electrophoresis, liquid chromatography-mass spectrometry and ion mass spectrometry. These methods require costly instruments, complicated sample operation and professional skills and take time despite high sensitivity and accuracy. Carbon quantum dots (CD), a new fluorescent nanomaterial, feature unique optical properties, desirable biocompatibility, green preparation processes and high quantum yields. Recent research shows that the CDs can be used as a fluorescence sensor for quantitative determination of TC based on an inner filter effect (IFE) caused by absorption of fluorophores excited and/or emitted by an absorbent in a detection system. However, due to a weak fluorescence quenching effect of TC on the CDs, the fluorescence intensity does not decrease obviously. Halloysite nanotubes (HNT), a silicate mineral, feature an abundant reserve, a large specific surface area and surface group abundance, and have unique mesoporous tubular cavities and modifiable internal and external surfaces. At present, a simple bottom-up method is generally used to synthesize a fluorescent HNT by in situ loading the carbon quantum dots on the surface of halloysite.

Bacillus anthracis, a dangerous bacterium that can cause anthrax, infects animals and humans through eating, breathing and skin contact, causes serious damage to skins, intestines and lungs of the infected ones, leads to anthrax infectious diseases, and in turn results in fatal infections in organisms. 2,6-dipicolinic acid (DPA), one of the main biomarkers of Bacillus anthracis, accounts for about 5%-15% of a dry weight of a Bacillus anthrax spore. DPA requires a long period and complicated operations when detected through a polymerase chain reaction (PCR) and an immunoassay. In contrast, fluorescence analysis can detect the Bacillus anthracis spore quickly, sensitively and highly selectively, with a low detection limit. However, a typical fluorescence detection and analysis method can merely detect a target by measuring change of a single fluorescence signal, and are highly susceptible to various irrelevant factors, causing sensitivity and selectivity to be reduced. A ratio fluorescence detection method can analyze a detected object by measuring relative change intensities of different fluorescence signals, effectively reduce interference of various kinds and improve a visualization degree. A luminescent lanthanide metal-organic framework (Ln-MOF) can be used as a feasible method.

The luminescent Ln-MOF is extensively applied to environmental pollution and biomolecule detection due to its unique optical properties, such as a large Stokes shift, a narrow emission band and a long wavelength. Since lanthanide ions have similar ionic radii and chemical behaviors, by doping terbium and europium into the same crystal equivalent metal positions of the MOF, two emission sources, that is, Eu³⁺ and Tb³⁺, can be generated as centers, which emit strong and visually detectable red light and green light respectively. Based on this, the luminescent Ln-MOF has been widely used in construction of luminescent materials.

In the luminescent Ln-MOF doped with terbium and europium, Tb³⁺ is rapidly complexed with DPA to detect the anthrax biomarkers. With a concentration increase of DPA, DPA can replace complexed water molecules, to effectively transfer energy to Tb³⁺, enhance an emission intensity of Tb³⁺, and produce strong and dominant green fluorescence. An europium ion complex has a strong red fluorescence emission peak at 616 nm. A diketone structure of TC molecules can chelate with Eu³⁺, transfer its excitation energy to Eu³⁺, and sensitize Eu³⁺ to emit light through an “antenna effect”. Based on the above technical principle, if the Ln-MOF can be combined with fluorescent HNT, a fluorescent probe can be formed to detect both TC and DPA simultaneously.

### SUMMARY

An objective of the present invention is to provide methods for preparing and using a dual-channel visual multicolor fluorescent probe for constructing a dual-channel fluorescence sensor based on in-situ synthesis of carbon dots on halloysite nanotubes (HNT) and loaded with a lanthanide metal-organic framework, which can implement rapid and simultaneous visual detection of DPA and TC.

In order to achieve the objective above, the present invention provides a method for preparing a dual-channel visual multicolor fluorescent probe.

A method for preparing a dual-channel visual multicolor fluorescent probe includes:
S1. performing amination modification of halloysite nanotubes, and specifically,
a. dispersing halloysite nanotubes into xylene, performing ultrasonication for a certain period of time, and stirring at a room temperature to make a solution disperse more evenly;
b. dropwise adding a silane coupler 3-chloropropyl trimethoxysilane into the solution above, stirring at a room temperature, and refluxing a mixture in an oil bath under a heating condition;
c. centrifuging the solution above, removing a supernatant, washing three times with ethanol, and vacuum drying under a heating condition;
d. dispersing all obtained products in deionized water, and stirring at a room temperature for a period of time after ultrasonication; and
e. adding polyethyleneimine, mixing and stirring for a period of time, refluxing a mixture in an oil bath under a heating condition, obtaining HNT-PEI through centrifuging, washing once with water, and drying in a vacuum drying oven to obtain HNT-PEI;
S2. synthesizing a blue carbon dot with a citric acid as a carbon source and synthesizing HNT@CDs, and specifically,
a. ultrasonically dispersing HNT-PEI and the citric acid in deionized water, and magnetically stirring for a period of time at a room temperature to uniformly mix HNT-PEI and the citric acid;
b. transferring an obtained solution to a polytetrafluoroethylene-lined autoclave, heating continuously for 12 hours, and synthesizing in situ the carbon dot on a surface of HNT through a hydrothermal reaction of PEI and the citric acid at a high temperature and a high pressure; and
c. after a reactor is cooled, removing a supernatant through centrifuging, washing a solid precipitate with absolute ethanol, and
vacuum drying to obtain HNT@CDs having blue fluorescence; and
S3. preparing HNT@CDs-MOF, and specifically,
a. ultrasonically dispersing HNT@CDs in deionized water;
b. dissolving a certain amount of Eu(NO₃)₃·6H₂O, Tb(NO₃)₃·6H₂O and anhydrous sodium acetate in deionized water, mixing with a solution above, dispersing 1,3,5-benzenetricarboxylic acid in ethanol, and dropwise adding a solution subjected to ultrasonic dissolution into a mixed solution, to form a layer of bimetal-organic framework (MOF) on a surface of HNT@CDs;
c. removing a supernatant through centrifuging, and washing with a mixed water and ethanol solution to remove free europium ions, terbium ions and sodium acetate; and
d. obtaining an HNT@CDs-MOF fluorescent nano-probe after vacuum drying under a heating condition.

A method for using a dual-channel visual multicolor fluorescent probe, where a detection method of DPA is included, and the method includes:
S1. dissolving DPA in deionized water to prepare a DPA solution having a certain concentration;
S2. buffering, for a constant volume, a certain amount of HNT@CDs-MOF fluorescent nano-probes with Tris-HCl having pH=8;
S3. adding different concentrations of DPA, and investigating sensitivity of the HNT@CDs-MOF fluorescent nano-probe to identify DPA in a channel having an excitation wavelength of Ex=280 nm; and
S4. implementing multi-color fluorescence semi-quantitative and qualitative detection of DPA with naked eyes under a 254 nm ultraviolet lamp.

A method for using a dual-channel visual multicolor fluorescent probe, where a detection method of tetracycline (TC) is included, and the method includes:
S1. dissolving TC in deionized water to prepare a TC solution having a certain concentration;
S2. buffering, for a constant volume, a certain amount of HNT@CDs-MOF fluorescent nano-probes with Tris-HCl having pH=9;
S3. dropwise adding a certain amount of TC, and measuring a fluorescence emission spectrum in a channel having an excitation wavelength Ex=375 nm; and
S4. implementing multi-color fluorescence semi-quantitative and qualitative detection of TC with naked eyes under a 365 nm ultraviolet lamp.

In this way, the present invention modifies the surface of halloysite, and prepares the dual-channel fluorescence sensor based on in-situ synthesis of carbon dots on halloysite nanotubes (HNT) and loaded with a lanthanide metal-organic framework, which can implement rapid and simultaneous visual detection of DPA and TC. A natural porous material is combined with the metal-organic framework, such that stability and detection sensitivity of the fluorescent probe are effectively improved, and the biomarkers can be rapidly, accurately and intuitively detected conveniently.

Technical solutions of the present invention will be further described below in detail with reference to accompanying drawings and in conjunction with examples.

### BRIEF DESCRIPTION OF THE DRAWINGS

FIG. 1 is a diagram showing a detection effect of an HNT@CDs-MOF probe on DPA according to Example 2 of the present invention; and

FIG. 2 is a diagram showing a detection effect of an HNT@CDs-MOF probe on TC according to Example 3 of the present invention.

### DETAILED DESCRIPTION OF THE EMBODIMENTS

Technical solutions of the present invention will be further described below with reference to accompanying drawings and in conjunction with examples.

### Example 1

A method for preparing a dual-channel visual multicolor fluorescent probe includes:

1. Amination Modification of Halloysite Nanotubes is Performed.

Amination modification of halloysite nanotubes is performed in a mode as follows. First, 1 g of halloysite nanotubes are dispersed into 50 mL of xylene, ultrasonication is performed for 15 minutes, and stirring is performed at a room temperature for 1 hour to make a solution disperse more evenly. 5 mL of silane coupler 3-chloropropyl trimethoxysilane is dropwise added into the solution above, stirring is performed at a room temperature for 10 minutes, a mixture is refluxed for 8 h in an oil bath at 80° C., and chloropropyl is introduced to surfaces of halloysite nanotubes through a coupling effect of the silane coupler and silicon hydroxyl groups on the surfaces of halloysite nanotubes. A supernatant is removed through centrifuging at 7,000 rmp for 3 min, an unreacted silane coupler is removed through washing three times with ethanol, and vacuum drying is performed for 5 hours at 60° C. Secondly, all obtained products are dispersed in 40 mL of deionized water, and stirring is performed at a room temperature for 30 min after ultrasonication, 2 g of polyethyleneimine (PEI) is added, mixing is performed and then stirring is performed for 30 min, a mixture is refluxed for 8 h in an oil bath at 90° C., and an amino group is introduced to the surfaces of halloysite nanotubes through covalent interaction between the amino group on a surface of PEI and chlorine on the surfaces of halloysite nanotubes. Then, HNT-PEI is obtained through centrifuging at 6,000 rmp for 3 min, washing is performed once with water, and then drying is performed in a vacuum drying oven at a set temperature of 60° C. to obtain HNT-PEI.

2. HNT@CDs is Synthesized.

A blue carbon dot is improved and synthesized with a citric acid as a carbon source. 500 mg of HNT-PEI and 300 mg of citric acids are ultrasonically dispersed in 15 mL of deionized water, and magnetically stirring is performed for 1 h at a room temperature to uniformly mix HNT-PEI and the citric acid. An obtained solution is transferred to a polytetrafluoroethylene-lined autoclave, heating is performed continuously for 12 hours at 160° C., and the carbon dot is synthesized in situ on a surface of HNT through a hydrothermal reaction of PEI and the citric acid at a high temperature and a high pressure. After a reactor is cooled, a supernatant is removed through centrifuging at 6,000 rmp for 3 min, a solid precipitate is washed three times with absolute ethanol, to remove unreacted organic matter and free carbon quantum dots, and vacuum drying is performed to obtain HNT@CDs having blue fluorescence.

3. HNT@CDs-MOF is Prepared.

100 mg of HNT@CDs is ultrasonically dispersed in 20 mL of deionized water. 8.82 mg (0.02 mmol) of Eu(NO₃)₃·6H₂O, 36.24 mg (0.08 mmol) of Tb(NO₃)₃ 6H₂O and 16.41 mg (0.02 mmol) of anhydrous sodium acetate are dissolved in 3 mL of deionized water, a solution above is mixing evenly, 1,3,5-benzenetricarboxylic acid is dispersed in 10 mL of ethanol, and a solution subjected to ultrasonic dissolution is dropwise added into a mixed solution, magnetically stirring is performed for 1 h at a room temperature, and a layer of bimetal-organic framework (MOF) is formed on a surface of HNT@CDs through a complexing effect of a carboxyl group and a rare earth ion. Then, a supernatant is removed through centrifuging, and washing is performed with a mixed water and ethanol solution to remove free europium ions, terbium ions and sodium acetate. An HNT@CDs-MOF fluorescent nano-probe is obtained after vacuum drying for 8 h at 60° C.

### Example 2

Bacillus anthracis (DPA) is Detected.

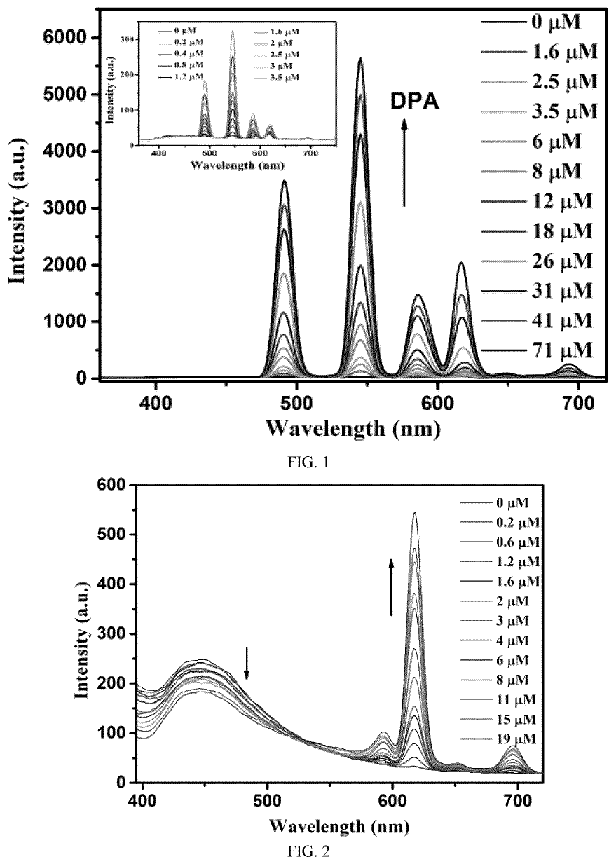

2,6-dipicolinic acid (DPA) is dissolved in deionized water to prepare a DPA solution (0 μm-81 μm) having a certain concentration, and 100 μL (1 mg/mL) HNT@CDs-MOF fluorescent nano-probes are buffered, for a constant volume of 2 mL, with Tris-HCl having pH=8. Different concentrations of DPA is added, and then sensitivity of the HNT@CDs-MOF fluorescent nano-probe to identify DPA is investigated in a channel having an excitation wavelength of Ex=280 nm. After DPA is added, a fluorescence intensity of the fluorescent nano-probe at 545 nm is significantly enhanced, and red fluorescence at 616 nm does not change significantly, as shown in FIG. 1. In the range of 0 μM-71 μM, with a concentration increase of DPA, red fluorescence changes to green fluorescence, and there is a desirable linear relationship between the concentration of DPA and a fluorescence intensity. A correlation coefficient R²=0.99784, and a related linear equation is I₅₄₅/I₆₁₆=3.54072C_{DPA}+0.44552. A detection limit is as low as 6.07 nM, and is significantly lower than an infection amount (60 μM) of a Bacillus anthracis spore.

According to sensitivity detection of the HNT@CDs-MOF fluorescent nano-probe to DPA, an emission spectrum and CIE coordinates of different concentrations of DPA in the channel having the excitation wavelength Ex=280 nm are measured, so as to confirm that the nano-sensor can change among various fluorescent colors in presence of DPA. Besides, multi-color fluorescence semi-quantitative and qualitative detection of DPA may be implemented with naked eyes under a 254 nm ultraviolet lamp.

### Example 3

Tetracycline (TC) is Detected.

TC is dissolved in deionized water to prepare a TC solution having a certain concentration, 100 L (1 mg/mL) of HNT@CDs-MOF fluorescent nano-probes are buffered, for a constant volume of 2 mL, with Tris-HCl having pH=9, a certain amount of TC (0 μM-19 μM) is added dropwise, and a fluorescence emission spectrum in a channel with an excitation wavelength Ex=375 nm is measured. A luminescence color changes from blue to red, and a fluorescence intensity at 616 nm of a system increases with the increase of TC, and a fluorescence intensity at 450 nm decreases. With a concentration increase of TC, a blue fluorescence intensity of the HNT@CDs-MOF nanosensor decreases slightly at 450 nm, and a red fluorescence intensity increases significantly at 616 nm, as shown in FIG. 2.

In ranges of 0 μM-6 μM and 6 μM-19 μM, there are desirable linear relationships between the concentration of TC and the fluorescence intensity, that is, R₁²=0.99761 and R₂²=0.98038, and related linear equations may be expressed as I₆₁₆/I_{45O}=0.28067C_{TC}+0.14139 and I₆₁₆/I₄₅₀=0.07941C_{TC}+1.31231. A detection limit is 11.31 nM, and is far below maximum residue limits (0.676 μM and 0.225 μM) of TC in milk as stipulated by the Food and Drug Administration (FDA) of the United States and the European Medicines Agency.

According to sensitivity detection of the HNT@CDs-MOF fluorescent nano-probe to TC, an emission spectrum and CIE coordinates of different concentrations of TC in the channel having the excitation wavelength Ex=375 nm are measured, so as to confirm that the nano-sensor can change among various fluorescent colors in presence of TC. Besides, multi-color fluorescence semi-quantitative and qualitative detection of TC may be implemented with naked eyes under a 365 nm ultraviolet lamp.

In this way, the present invention provides a technical solution for preparing the dual-channel fluorescent nano-probe that is based on in-situ growth of the carbon dot on halloysite nanotubes and doping on the lanthanide bimetal-organic framework and has sensitivity, a wide range and desirable selectivity, which can implement rapid and simultaneous visual detection of DPA and TC. Under the excitation wavelength of 280 nm, the fluorescent nano-sensor can specifically identify DPA. After DPA is complexed with Tb³⁺, the fluorescent nano-sensor emits feature green light having a wavelength of 545 nm through an antenna effect, and the fluorescence changes from red to green, thus implementing rapid visual detection of DPA. At the excitation wavelength of 375 nm, the fluorescent nano-sensor can specifically identify TC and changes from blue fluorescence to red fluorescence. After TC is complexed with Eu³⁺, the fluorescent nano-sensor emits a feature red light having a wavelength of 616 nm through the antenna effect, and the blue fluorescence emission intensity (450 nm) of halloysite nanotubes loaded with the carbon dot decreases slightly with the increase of tetracycline, and change in the ratio of red emission to blue emission of the Eu-TC complex is recorded, and the highly sensitive detection of tetracycline is implemented. In the range of 0 μM-71 μM of DPA, the detection limit of the fluorescent sensor to DPA is as low as 6.07 nM. In the range of 0 μm-19 μm of TC, the detection limit of the fluorescent sensor to TC is as low as 11.31 nM, accurate detection of DPA and TC is implemented and detection requirements of TC and DPA in food and environmental samples can be satisfied.

Finally, it should be noted that the examples above are merely used to describe the technical solution of the present invention rather than limit same. Although the present invention has been described in detail with reference to the preferred example, those skilled in the art should understand that modifications or equivalent replacements can be made to the technical solution of the present invention, and these modifications or replacements cannot make the modified technical solution deviate from the spirit and scope of the technical solution of the present invention.

What is claimed is:

1. A method for preparing a dual-channel visual multicolor fluorescent probe, comprising: S1. performing amination modification of halloysite nanotubes, comprising
a. dispersing the halloysite nanotubes into xylene, performing ultrasonication for a predetermined period of time to obtain a solution, and stirring at a room temperature to make the solution disperse more evenly;
b. dropwise adding a silane coupler 3-chloropropyl trimethoxysilane into the solution above, stirring at the room temperature to obtain a mixture, and refluxing the mixture in an oil bath under a heating condition to obtain a solution;
c. centrifuging the solution above, removing a supernatant, washing three times with ethanol, and vacuum drying under a heating condition;
d. dispersing all obtained products in deionized water, and stirring at the room temperature for a period of time after the ultrasonication; and
e. adding polyethyleneimine, mixing and stirring for a period of time to obtain a mixture, refluxing the mixture in an oil bath under a heating condition, obtaining HNT-PEI through centrifuging, washing once with water, and drying in a vacuum drying oven to obtain HNT-PEI;
S2. synthesizing a blue carbon dot with a citric acid as a carbon source and synthesizing HNT@CDs, comprising
a. ultrasonically dispersing HNT-PEI and the citric acid in deionized water, and magnetically stirring for a period of time at the room temperature to uniformly mix HNT-PEI and the citric acid to obtain a solution;
b. transferring the solution to a polytetrafluoroethylene-lined autoclave, heating continuously for 12 hours, and synthesizing in situ the blue carbon dot on a surface of HNT through a hydrothermal reaction of PEI and the citric acid at a high temperature and a high pressure; and
c. after a reactor is cooled, removing a supernatant through centrifuging, washing a solid precipitate with absolute ethanol, and vacuum drying to obtain HNT@CDs having blue fluorescence; and
S3. preparing HNT@CDs-MOF, comprising
a. ultrasonically dispersing HNT@CDs in deionized water to obtain a solution;
b. dissolving a predetermined amount of Eu(NO₃)₃·6H₂O, Tb(NO₃)₃·6H₂O and anhydrous sodium acetate in deionized water, mixing with the solution above, dispersing 1,3,5-benzenetricarboxylic acid in ethanol, and dropwise adding a solution subjected to ultrasonic dissolution into a mixed solution, to form a layer of bimetal-organic framework (MOF) on a surface of HNT@CDs;
c. removing a supernatant through centrifuging, and washing with a mixed water and ethanol solution to remove free europium ions, terbium ions and sodium acetate; and
d. obtaining an HNT@CDs-MOF fluorescent nano-probe after vacuum drying under a heating condition.

2. The method for preparing the dual-channel visual multicolor fluorescent probe according to claim 1, wherein a detection method of Bacillus anthracis (DPA) is comprised, and the detection method comprises:
S1. dissolving DPA in deionized water to prepare a DPA solution having a predetermined concentration;
S2. buffering, for a constant volume, a predetermined amount of HNT@CDs-MOF fluorescent nano-probes with Tris-HCl having pH=8;
S3. adding different concentrations of DPA, and investigating sensitivity of the HNT@CDs-MOF fluorescent nano-probe to identify DPA in a channel having an excitation wavelength of Ex=280 nm; and
S4. implementing a multi-color fluorescence semi-quantitative and qualitative detection of DPA with naked eyes under a 254 nm ultraviolet lamp.

3. The method for preparing the dual-channel visual multicolor fluorescent probe according to claim 1, wherein a detection method of tetracycline (TC) is comprised, and the detection method comprises:
S1. dissolving TC in deionized water to prepare a TC solution having a predetermined concentration;
S2. buffering, for a constant volume, a predetermined amount of HNT@CDs-MOF fluorescent nano-probes with Tris-HCl having pH=9;
S3. dropwise adding a predetermined amount of TC, and measuring a fluorescence emission spectrum in a channel having an excitation wavelength Ex=375 nm; and
S4. implementing a multi-color fluorescence semi-quantitative and qualitative detection of TC with naked eyes under a 365 nm ultraviolet lamp.
